(19) United States
(12) Patent Application Publication
(10) Pub. No.: US 2024/0352393 A1
(43) Pub. Date: Oct. 24, 2024

(54) A SYSTEM AND METHOD FOR ENHANCING GAS MASS TRANSFER

(71) Applicant: RAINFOREST ALGAE CORP., Calgary (CA)

(72) Inventor: Stanley PANKRATZ, Calgary (CA)

(21) Appl. No.: 18/686,062

(22) PCT Filed: Aug. 23, 2022
(86) PCT No.: PCT/CA2022/051276

§ 371 Date: Feb. 23, 2024

Related U.S. Application Data
(60) Provisional application No. 63/236,123, filed on Aug. 23, 2021.

(51) Int. Cl. C12M 3/04; C12M 1/00; C12M 1/107

(52) U.S. Cl. CPC C12M 27/12; C12M 21/04; C12M 29/08

(57) ABSTRACT

The present invention provides a system and a method for enhancing mass transfer of a gas between a gaseous medium and a liquid medium in reactions involving such a mass transfer, and for effective mixing of the liquid medium.

### FIELD

The present invention relates generally to the cultivation of biomass producing microorganisms. In particular, the present disclosure relates to an improved system and method for enhancing mass transfer of gas(es) between gaseous and liquid media, in reactions involving mass gas transfer.

### BACKGROUND

Many reactions/processes rely on the transfer of gases, a physical process that often affects reaction rates. Examples of such reactions include cultivation of microorganisms, CO₂ capture, waste water treatment processes, etc.

Microalgae and cyanobacteria are renewable, sustainable, and economical sources of biofuels, bioactive medicinal products, and food ingredients. Microalgae along with cyanobacteria produce approximately half of the atmospheric oxygen and use simultaneously the greenhouse gas carbon dioxide to grow photoautotrophically. Due to their economic value and social benefits, extensive efforts have been made to achieve cost effective microalgae biomass production with enhanced growth rate, culture densities and product yield.

Conventionally, open raceway-ponds have been purported as the most cost-effective cultivation design for achieving favourable net present values for desired products. These are shallow ponds that circulate a volume of nutrient rich water generally by means of a paddle wheel. These types of ponds require large tracts of land, large amounts of water, and require considerable amounts of energy for cultivation, harvesting and product processing. However, yields tend to be relatively low. They are generally limited to warm geographic areas that have high solar irradiance. Gas transfer between the atmosphere and the liquid media is sub-optimal, and they are prone to contamination, fouling, culture crashes and require considerable maintenance.

Bioreactors are fabricated devices and systems that environmentally control and support a biologically active growth cultures, which can be aerobic or anaerobic. In each case chemical processes and transformations take place involving chemistries, and/or organisms, cells and tissues generally involving gas and liquid phases. Generally, reactors are designed to maximize the net present value of a particular process to achieve with greatest efficiency a desired output product, providing the greatest yield of the desired product while requiring the least amount of capital investments and operating costs.

Column photobioreactors with air sparging systems achieve significantly higher algae culture densities and yields, and have the advantage of a closed and controlled environment. They involve higher capital costs than raceway pond systems and are more costly to operate and maintain. They also struggle with issues related to fouling and mass transfer of gases.

Similarly, some of the tubular photobioreactors have achieved some of the highest recorded algae culture densities and yields through highly controlled environments. They again involve high capital, operating and maintenance costs. In a similar way to the experiences found with column photobioreactors, mass transfer of gases, maintenance and energy challenges remain.

En masse, photobioreactor designs have failed to adequately improve algae growth rates and densities, thus making it challenging to achieve a return on investment on upfront capital costs and production cost reductions necessary to be economically viable unless producing very high value products from the biomass. Therefore, utility is limited.

The majority of the open environment cultivation (such as in ponds) or closed bioreactor-based cultivation of microalgae operate in a “suspension culture mode” wherein the algal cells are dispersed in a liquid medium/water. Recently, some efforts have been made involving “attachment culture mode”, wherein the microalgae cells create biofilms, adhering to the surface of solid or semi-solid substrate materials for growth.

The limitations in achieving higher growth rates and culture densities with the currently known systems may be due to the fact that they have been predominantly focused on photon flux or the ability of the light to penetrate the dense cell culture, thereby providing the energy to drive photosynthetic activity. Hence, bioreactor designs have focused on enhancing light penetration with the culture media. Culturing biofilms on substrates versus within a circulating media have been known to produce extraordinary culture densities, yet are limited by light penetration and exchange of nutrients and metabolites, therefore necessitating harvesting via scraping layers of the growing biomass to ensure that deeper layers receive light energy and nutrients for growth.

Therefore, there remains a need for a cost-effective system/method for improving reactions involving mass transfer of gas(s) between gaseous and liquid media, such as cultivation of microorganisms, direct air capture of gases, waste water treatment, etc.

### SUMMARY OF THE INVENTION

The present invention relates to a system and method for enhancing mass transfer of a gas between a gaseous medium and a liquid medium in reactions involving such a mass transfer.

In accordance with an aspect of the present invention, there is provided a system for enhancing a reaction involving mass transfer of a gas between a gaseous medium and a liquid medium in a reservoir, the system comprising: a) means to provide a gaseous medium in contact with the liquid medium; and b) one or more movable members, each having a surface, the one or more members configured to periodically expose at least a portion of said surface to the gaseous medium and the liquid medium to form a wetted surface, thereby generating a renewing wetted surface for enhancing mass transfer of the gas between the liquid medium and the gaseous medium.

In accordance with another aspect of the present invention, there is provided a method of enhancing a reaction involving mass transfer of a gas between a gaseous medium and a liquid medium, the method comprising: a) providing the liquid medium and the gaseous medium in contact with a surface one or more movable members configured to periodically expose at least a portion of said surface to the gaseous medium and the liquid medium, and b) exposing said surface of the one or more movable members periodically to the gaseous medium and the liquid medium to form a renewing wetted surface, thereby enhancing mass transfer of the gas between the liquid and gaseous medium.

In accordance with another aspect of the present invention, there is provided use of the system described herein for cultivating microorganisms.

### BRIEF DESCRIPTION OF THE FIGURES

Further features and advantages of the present improvements/invention will become apparent from the following detailed description, taken in combination with the appended figures, in which:

FIG. 1 is a schematic partial view of the system in accordance with an embodiment of the present invention.

FIG. 2 is a schematic partial view of the system in accordance with an embodiment of the present invention.

### DETAILED DESCRIPTION

Unless defined otherwise, all technical and scientific terms used herein have the same meaning as commonly understood by one of ordinary skill in the art to which this invention belongs.

Unless the context requires otherwise, throughout this specification and claims, the words “comprise”, “comprising” and the like are to be construed in an open, inclusive sense. The words “a”, “an”, and the like are to be considered as meaning at least one and not limited to just one.

The term “nanobubbles” as used herein refers to ultra-fine bubbles, nanopores, nanostructures, and/or nanoporous liquids which have bubble diameters/sizes of less than 10⁻⁶ m.

As used herein, the term “about” refers to approximately a +/−10% variation from a given value. It is to be understood that such a variation is always included in any given value provided herein, whether or not it is specifically referred to.

The present invention provides a system and method for enhancing mass transfer of gas(es) between gaseous and liquid media, which in turn results in improving/enhancing reactions involving such mass gas transfer.

The present application has established that by providing a movable member having a surface configured to be periodically/alternately exposed to the gaseous medium and the liquid medium forms, a renewing thin wetted surface outside the liquid medium, which provides a larger, more effective relative surface area (beyond the stationary gas-liquid interface), which in turn increases the potential for increasing the rate at which the gas may become solubilized or exchanged within the liquid phase, and enhances mass transfer of gases between the gaseous and liquid phases, thereby leading to the potential for higher reaction rates and/or higher product yields. The system also provides effective mixing within the liquid medium.

Furthermore, the orientation of the central axis of the rotatable member at angles between 0 or 90 degrees, creates a circular third dimension for mixing within the liquid and gas phases, and changes the amount of wetted surface area available for gas transfer between the liquid and gaseous phase to take place.

It has also been found that the incorporation of nanobubbles in the liquid medium further increases the interface surface area that enables more efficient mass transfer of gases between the liquid and gas phases in a solution. It is believed that gas nanobubbles found within the wetting surface passing out of the liquid phase on the rotating surface, an effective further mass transfer of gas exchange that takes place that is dependent on the partial pressures of the gases in the gas phase and their affinity to enter or leave the liquid phase.

The transfer of gas(es) between the thin film of solution including nanobubbles on the moving/rotating surfaces while traveling above the more stationary liquid phase can be extremely efficient because of the renewing and circulating large mass surface areas which enable the effective mass transfer of gases between liquid and gas phases.

Furthermore, the nanobubbles found within the liquid phase provide enhanced light scattering within the liquid phase and throughout the liquid media to enhance photosynthetic activity.

The system and method of the present invention can shift gas saturation limits in the liquid medium through the use of generated nanobubbles included in the liquid phase to increase the rates of reactions and biological metabolic activity.

The system and method of the present invention can be applied for any chemical and biological process relying on the presence or storage of gas in solution, wherein the mass transfer of gases between the liquid and gas phases may be improved, such as microorganism cultivation (such as microalgae, cyanobacteria, yeast, etc.) with enhanced reaction rate, product yield, and/or density.

The present application has also established that by providing a transparent, movable surface, configured to prevent adherence of microorganisms or other reaction products thereto, and configured to be periodically/alternately exposed to the gaseous medium and the liquid cultured medium, can lead to further enhance growth rates, improve densities and/or yields of autotrophically grown microorganisms, such as algae/cyanobacteria cultures.

In one aspect, the present invention provides a system for enhancing a reaction involving a mass transfer of a gas between a gaseous medium and a liquid medium in a reservoir. The one or more movable members are configured to periodically/alternately expose a surface thereof, to the gaseous medium and the liquid medium to form a wetted surface upon movement of the movable member leaving the liquid media and entering the gas medium, thereby generating a renewing wetted surface for enhancing mass transfer of the gas between the liquid and gaseous media.

The reservoir can be a container, receptacle, or a body of water (such as such as pond, lake, channel, stream, etc.).

The one or more movable members can be rotatable members having a central axis oriented at an angle from 0° to 90° relative to horizontal, and configured to rotate about the central axis, or one or more movable tracks.

In some embodiments, the central axis of the rotatable member is oriented at an angle between 0° to 90° relative to horizontal. In some embodiments, the central axis of the rotatable member is oriented at an angle between 0° to 75° relative to horizontal.

Non-limiting examples of a rotatable member include a closable/sealable container, a cylinder, a shaft, an auger, etc. Non-limiting example of movable track includes conveyor belts.

In some embodiments, the reservoir is a closable/sealable container, provided with inlet(s) for the gaseous and liquid medium and outlet for the reaction products, wherein the container itself is rotatable on its central axis, and the renewing wetted surface is the interior surface of the container. The container can have any shape such as cylindrical, elliptical, round, square, rectangular, etc. (preferably cylindrical).

In some embodiments, the reservoir is a receptacle, wherein the rotatable member is configured to be positioned in the receptacle, and the renewing wetted surface is an outer and/or inner surface of the rotatable member. The receptacle can be closable receptacle, such as a closable container, or an open receptacle, such as trough.

The liquid medium can comprise aqueous and/or non-aqueous liquids, for example water, hydrocarbons, etc. The liquid medium can comprise one liquid or a mixture of two or more liquids. The gaseous medium can comprise one gas or a mixture of two or more gases.

In some embodiments, the reservoir is a body of water (such as pond, lake, channel, stream, etc.) wherein the rotatable member is configured to be positioned in the reservoir, and the wetted surface can be an outer and/or inner surface of the rotatable member.

In some embodiments, the rotatable member is a closable/sealable container, a cylinder, a shaft, or an auger, rotating on an axis. In some embodiments, the shaft further comprises one or more discs (optionally rotatable) mounted on the shaft.

The closable/sealable container can comprise closure cap and/or a sealing member such as a gasket for operably sealing the container.

In some embodiments, the movable member is a movable track (such as, a conveyor belt) configured to move in and out of the liquid medium.

In some embodiments, the closable/sealable container of present invention further comprises a secondary rotatable member, preferably having a rotating axis coaxial with the central axis of the container wherein a surface of the secondary rotatable member is also exposed periodically/alternately to the gaseous medium and the liquid medium upon rotation of the rotatable member.

In some embodiments, the secondary rotatable member is a cylinder, an auger or a shaft optionally comprising one or more discs (optionally rotatable) mounted thereon. At least one surface of the secondary rotatable member provides additional surface upon which “liquid” wetting may occur, thus providing additional surface area upon which gas transfer may take place between the gaseous and the liquid media, and potentially aid in mixing.

In some embodiments, the secondary rotatable member comprises at least one additional closed/sealed container placed inside the rotatable container, each containing the liquid medium and the gaseous medium, and each preferably having a rotating axis coaxial with the central axis of the container. In such embodiments, the inner and outer surfaces of the additional container provide additional surfaces upon which “liquid” wetting may occur, thereby further enhancing the gas mass transfer.

In some embodiments, the liquid medium comprises nanobubbles of the gas. The presence of the nanobubbles may further increase the surface upon which mass gas transfer can take place within the liquid phase.

In some embodiments, the surface(s) involved in the formation of renewing wetted surface are textured, corrugated or coated to increase the effective relative surface area to further enhance the gas transfer rate.

The system comprises means to supply liquid and/or gaseous medium to the reservoir.

In some embodiments, the gaseous medium is provided above the upper surface of the liquid medium.

The rotation of the rotatable member can be achieved directly or indirectly via a motor or one or more drive mechanisms known in the art. In some embodiments, the system comprises means/regulators for adjusting rotation speed of the rotatable member. Different gases will have differing solubility within the liquid medium and differing surface adsorption behaviors. Changing the rate of rotation of the moving surfaces may impact the rate at which the gas is transferred between the liquid and gas phases and the rate of mixing within the solution.

In some embodiments, the system comprises a temperature sensor (e.g. a thermocouple for sensing temperature in the gaseous and liquid phases. In some embodiments, the system comprises a pressure sensor for sensing pressure in the gaseous and liquid phases. In some embodiments, the system further comprises regulators for adjusting temperature and/or pressure in the gaseous and liquid phases.

In another aspect, the present invention provides method of enhancing a reaction involving mass transfer of a gas between a gaseous medium and a liquid medium. The method comprises providing the liquid medium and the gaseous medium in contact with a surface of one or more movable members configured to periodically/alternately expose at least a portion of the surface to the gaseous medium and the liquid medium, and exposing the movable surface of the one or more movable members periodically/alternately to the gaseous medium and the liquid medium to form a renewing wetted surface, thereby enhancing mass transfer of the gas between the liquid and gaseous medium.

The one or more movable members can be rotatable members having a central axis, and configured to rotate about the central axis, or one or more movable tracks, such as conveyor belts, as discussed above.

In some embodiments, the movable member is a closable/sealable container configured to rotate about the central axis thereof, and the method comprises providing the liquid medium and the gaseous medium in the closable/sealable container, wherein the renewing wetted surface is the interior surface of the container that is exposed periodically/alternately to the gaseous medium and the liquid medium upon rotation of the container.

In some embodiments, the movable member is a rotatable member configured to rotate about the central axis thereof, and the method comprises providing the liquid medium in a receptacle comprising the movable member, wherein the renewing wetted surface is at least the outer surface of the rotatable member that is exposed periodically/alternately to the gaseous medium and the liquid medium upon rotation of the rotatable member.

In some embodiments, the reservoir is a body of water (such as pond, lake, channel, stream, etc.), and the movable member is a rotatable member configured to rotate about the central axis thereof, and the method comprises providing the rotatable member in contact with water, wherein the renewing wetted surface is at least the outer surface of the rotatable member that is exposed periodically/alternately to the gaseous medium and the liquid medium upon rotation of the rotatable member.

In some embodiments, the movable member is a moving track, and the method comprises providing the liquid medium in a receptacle comprising the moving track, wherein the renewing wetted surface is the surface of the track that is exposed periodically/alternately to the gaseous medium and the liquid medium upon rotation of the rotatable member.

In some embodiments, the reservoir is a body of water (such as pond, lake, channel, stream, etc.), the movable member is a moving track, and the method comprises providing the movable track in contact with water, wherein the renewing wetted surface is the surface of the track that is exposed periodically/alternately to the gaseous medium.

The system and method of the present system can be used for any chemical, biological, or engineering process relying on the mass transfer of gas between liquid and gas phases.

In some embodiments, the system of the present invention is for cultivation of microorganisms (such as microalgae, cyanobacteria, yeast, etc.). In such embodiments, the liquid medium is provided with a microorganism culture and nutrients.

The microorganism can be autotrophic organism or heterotrophic organism. Autotrophic organisms, such as microalgae and cyanobacteria, grow by utilizing photosynthesis, a process that relies upon the exchanges of CO₂ and O₂ between the liquid and gas phases. Heterotrophic organisms rely on organic substances for their primary energy requirements. Such organisms are involved in fermentation. In each case, these processes rely on mass gas transfer processes between phases which may be enhanced by the processes associated with this system.

In such embodiments, the movable member is configured to prevent adherence of the microorganism or other reaction products on the surface being periodically/alternately exposed to the gaseous medium and the liquid medium.

In some embodiments, the adherence of microorganisms or other reaction products is prevented by adjusting rotation speed of the rotational member, by adjusting speed of the moving tack, and/or by rendering the surface repelling to the microorganisms/other products by use of a suitable material and/or a suitable coating.

The system and method of the present invention can also be applied for other processes that rely on mass gas transfers between liquid and gas phases, such as direct air capture techniques for capturing CO₂ or other gaseous products.

Many energy intensive waste water treatment processes rely on mass gas transfer between liquid and gas phases and would benefit from the system and method of the present invention.

To gain a better understanding of the invention described herein, the following example(s) are set forth. It will be understood that this example is intended to describe an illustrative embodiment of the invention and is not intended to limit the scope of the invention in any way.

### Examples

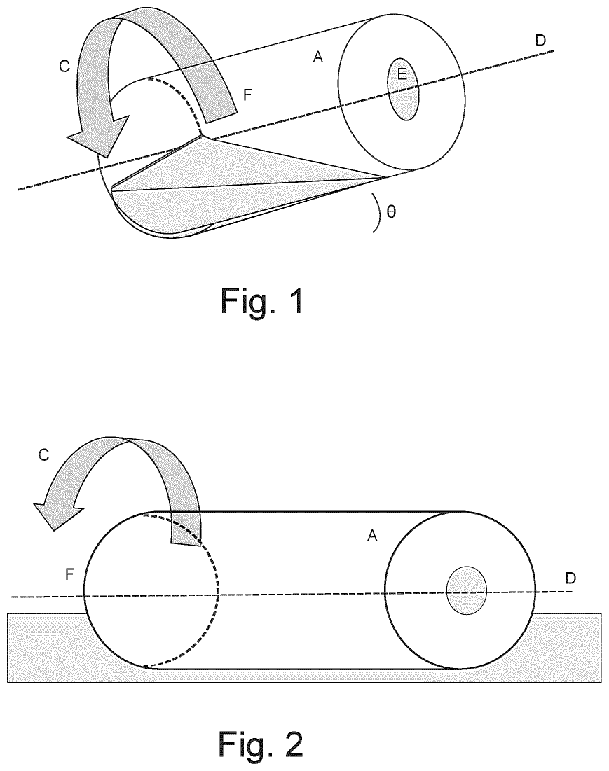

FIG. 1 schematically illustrates an example of the system of the present invention for cultivation of microorganisms, which comprises a rotatable cylindrical container A partially provided with a liquid medium B and gas F above the liquid. The container has a central axis D oriented at an angle θ between 0° and 90° relative to horizontal and is configured to rotate about its axis D in a direction C, via a drive mechanism (not shown) so that its inside surface is alternatingly immersed in the liquid and then the wetted surface is exposed to the gas. The container has a closable/sealable opening E which provides inlet for the liquid and the gas, as well as outlet for the reaction products, and also allows for control of the temperature and pressure of the liquid and gas in the container.

FIG. 2. Schematically illustrates an example of the system of the present invention for cultivation of microorganisms, which comprises a rotatable cylinder A, partially immersed horizontally in a liquid B, with air or gas phase F above the liquid. The cylinder has a central axis D, and configured to rotate about its horizontal axis in direction C via a drive mechanism (not shown) so that the exterior surface of the cylinder is alternatingly immersed in the liquid and then the wetted surface is exposed to the gas phase F. The liquid is provided in a receptacle or the liquid is water in a reservoir.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

We claim:

1. A system for enhancing a reaction involving mass transfer of a gas between a gaseous medium and a liquid medium in a reservoir, the system comprising:
a) means to provide a gaseous medium for contact with the liquid medium; and
b) one or more movable members, each having a surface, the one or more movable members configured to periodically expose at least a portion of said surface to the gaseous medium and the liquid medium to form a wetted surface, thereby generating a renewing wetted surface for enhancing mass transfer of the gas between the liquid medium and the gaseous medium.

2. The system of claim 1, wherein one or more movable members are rotatable members having a central axis oriented at an angle from 0° to 90° relative to horizontal, the rotatable member being configured to rotate about the central axis.

3. The system of claim 1, wherein the reservoir is a sealable container, the sealable container being the rotatable member, wherein the sealable container is provided with an inlet for the gaseous and liquid medium and an outlet for the reaction products, and the renewing wetted surface is the interior surface of the sealable container.

4. The system of claim 3, wherein the container further comprises a secondary rotatable member, preferably having a rotating axis coaxial with the central axis of the container.

5. The system of claim 4, wherein the secondary rotatable member is a container, a cylinder, a shaft, or an auger.

6. The system of claim 5, wherein the shaft further comprises one or more discs (optionally rotatable) mounted thereon.

7. The system of claim 4, wherein the secondary rotatable member comprises one or more additional closed/sealed containers, each containing the liquid medium and the gaseous medium, and each preferably having a rotating axis coaxial with the central axis of the container.

8. The system of claim 2, wherein the reservoir is a receptacle or a body of water, and wherein the one or more rotatable members are selected from a container, a cylinder, a shaft, or an auger, wherein the renewing wetted surface is an outer surface of the rotatable member, and optionally an inner surface of the rotatable member.

9. The system of claim 8, wherein the rotatable member further comprises one or more discs (optionally rotatable) mounted on the shaft.

10. The system of claim 1, wherein the movable member is a movable track.

11. The system of claim 10, wherein the reservoir is a receptacle or a body of water.

12. The system of any one of claims 1 to 11, wherein the liquid medium comprises nanobubbles of the gas.

13. The system of any one of claims 1 to 12, wherein the system is for cultivation of microorganism (such as microalgae, cyanobacteria, yeast), and wherein the liquid medium comprises microorganism culture and nutrients.

14. The system of claim 13, wherein the rotatable member is configured to prevent adherence of the microorganism and/or other reaction products on the surface being periodically/alternately exposed to the gaseous medium and the liquid medium.

15. The system of any one of claims 1 to 14, wherein the system is for capturing gases via direct air capture process, or for treating waste water.

16. A method for enhancing a reaction involving mass transfer of a gas between a gaseous medium and a liquid medium, the method comprising:
a) providing the liquid medium and the gaseous medium in contact with a surface one or more movable members configured to periodically expose at least a portion of said surface to the gaseous medium and the liquid medium, and
b) exposing said surface of the one or more movable members periodically to the gaseous medium and the liquid medium to form a renewing wetted surface, thereby enhancing mass transfer of the gas between the liquid and gaseous medium.

17. The method of claim 16, wherein the movable member is a rotatable member having a central axis oriented at an angle between 0° and 90° relative to horizontal, the rotatable member being configured to rotate about the central axis.

18. The method of claim 17, wherein the rotatable member is a sealable container configured to rotate about the central axis thereof, and the method comprises providing the liquid medium and the gaseous medium in the sealable container, wherein the renewing wetted surface is the interior surface of the container that is exposed periodically to the gaseous medium and the liquid medium upon rotation of the container.

19. The method of claim 17, comprising providing the liquid medium and the gaseous medium in a receptacle comprising the rotatable member, wherein the renewing wetted surface is the outer surface of the rotatable member that is exposed periodically to the gaseous medium and the liquid medium upon rotation of the rotatable member.

20. The method of claim 17, wherein the liquid medium is body of water, and the method comprises providing the rotatable member in contact with the water and the gaseous medium, wherein the renewing wetted surface is the outer surface of the rotatable member that is exposed periodically/alternately to the gaseous medium and the liquid medium upon rotation of the rotatable member.

21. The method of claim 19 or 20, wherein the rotatable member is a container, a cylinder, a shaft, or an auger.

22. The method of any one of claims 16 to 21, wherein the liquid medium comprises nanobubbles of the gas.

23. The method of claim 16, wherein the movable member is a movable track, and the method comprises providing the liquid medium in a receptacle comprising the movable member, wherein the renewing wetted surface is formed on the surface of the track that is exposed periodically to the gaseous medium and the liquid medium.

24. The method of any one of claims 16 to 23, wherein the method comprises cultivation of a microorganism, and wherein the liquid medium is provided with a microorganism culture and nutrients.
